(19) United States
(12) Patent Application Publication
(10) Pub. No.: US 2010/0297110 A1
(43) Pub. Date: Nov. 25, 2010

(54) ANTIBODY SPECIFIC FOR HUMAN IL-4 FOR THE TREATMENT OF CANCER

(75) Inventors: Thomas Hoeger, Laudenbach (DE); Juergen Gamer, Dossenheim (DE); Giorgio Stassi, Palermo (IT); Matilde Todaro, Palermo (IT)

Correspondence Address: DUANE MORRIS LLP - (prev. San Francisco);IP DEPARTMENT, 30 SOUTH 17TH STREET, PHILADELPHIA, PA, 19103-4196, US

(73) Assignee: APOGENIX GMBH, Heidelberg (DE)

(21) Appl. No.: 12/293,781

(22) PCT Filed: Mar. 21, 2007
(86) PCT No.: PCT/EP2007/002497

§ 371 Date: Aug. 9, 2010

(30) Foreign Application Priority Data
Mar. 22, 2006 (EP) 06 005 894.8

(51) Int. Cl. A61K 39/395; C07K 16/24; A61P 35/00

(52) U.S. Cl. 424/132.1; 530/389.2; 530/388.23; 530/387.3; 424/145.1; 424/158.1; 424/135.1; 424/136.1; 424/133.1

(57) ABSTRACT

The present invention relates to the use of an antibody or an antigen-binding fragment thereof with specific binding activity for human interleukin-4 for the prevention and/or treatment of cancer.

The present invention relates to the use of an antibody or an antigen-binding fragment thereof with specific binding activity for human interleukin-4 for the prevention and/or treatment of cancer.

WO 2004/069274 refers to the use of cytokine antagonists which modulate the expression and/or the function of a cytokine for the down-regulation of an anti-apoptotic protein in a cell. In particular, it is referred to the use of cytokine antagonists for the treatment of cancer. Antibodies directed against cytokines are indicated as examples of cytokine antagonists.

The European patent application EP-A-0 730 609 describes fusion proteins, antibodies and fragments thereof characterized by a dissociation constant equal to or less than 2×10⁻¹⁰ M for human IL-4. In particular, the monoclonal antibodies 3B9 and 6A1 are disclosed. Thus, antibodies are indicated as being suitable for the treatment and/or prevention of allergic conditions. The treatment of cancer with such monoclonal antibodies, however, is neither disclosed nor suggested.

It was found that the anti-IL-4 antibodies disclosed in European patent EP-A-0 730 609 are especially suitable for the treatment of cancer diseases. Thus, the present invention refers to the use of antibodies or antigen-binding fragments specific for human interleukin-4 for the manufacture of a medicament for the prevention and/or treatment of cancer, wherein the antibodies and antibody fragments are characterized by a dissociation constant equal to or less than 2×10⁻¹⁰ M for human IL-4. The dissociation constant and specificity of the antibody may be determined as described in EP-A-0 730 609.

A first preferred embodiment relates to the use of
(i) an antibody or an antigen-binding fragment specific for human interleukin-4, wherein said antibody comprises at least one heavy chain variable region and at least one light chain variable region, wherein the amino acid sequence of the complementarity-determining regions (CDRs) of the heavy chains are:

(SEQ ID No. 1)
(a) Thr Ser Gly Met Gly Val Ser;
(SEQ ID No. 2)
(b) His Ile Tyr Trp Asp Asp Asp Lys Arg Tyr Asn
Pro Ser Leu Lys Ser;
(SEQ ID No. 3)
(c) Arg Glu Thr Val Phe Tyr Trp Tyr Phe Asp Val;

and
(d) a sequence derived by substituting 1, 2 or 3 amino acids of SEQ ID Nos. 1, 2 and/or 3;
   and/or the amino acid sequences of the complementary determining regions (CDRs) of the light chain are:

(SEQ ID No. 4)
(a) Lys Ala Ser Gln Ser Val Asp Tyr Asp Gly Asp
Ser Tyr Met Asn;
(SEQ ID No. 5)
(b) Ala Ala Ser Asn Leu Glu Ser;
(SEQ ID No. 6)
(c) Gln Gln Ser Asn Glu Asp Pro Pro Thr (Arg);

(d) a sequence derived by substituting 1, 2 or 3 amino acids of SEQ ID Nos. 4, 5, and/or 6;
   or
(ii) an antibody or an antigen-binding fragment thereof which recognizes the same epitope on human IL-4 as the antibody of (i);
   for the manufacture of a medicament for the prevention and/or treatment of cancer.

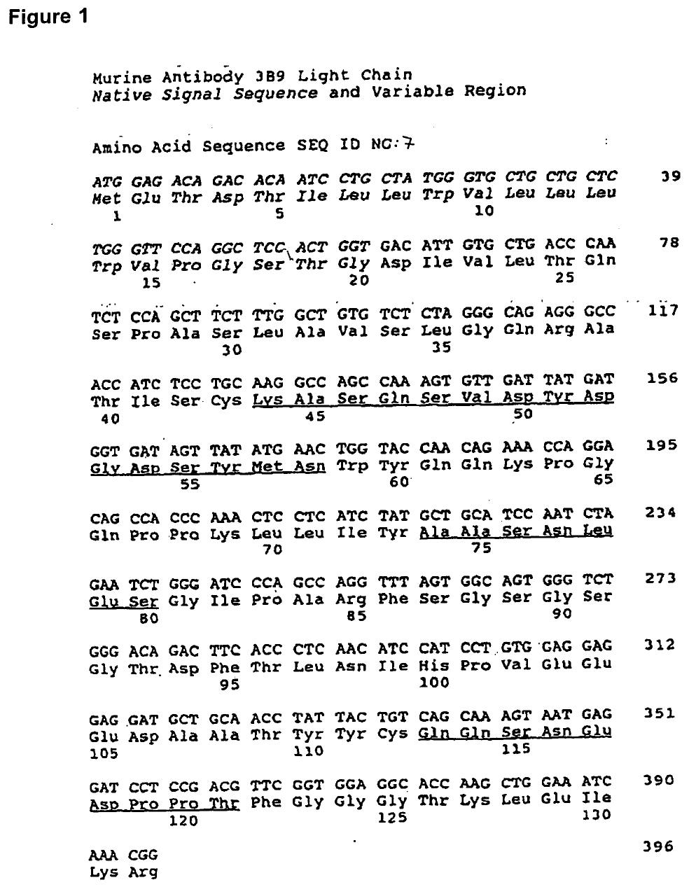

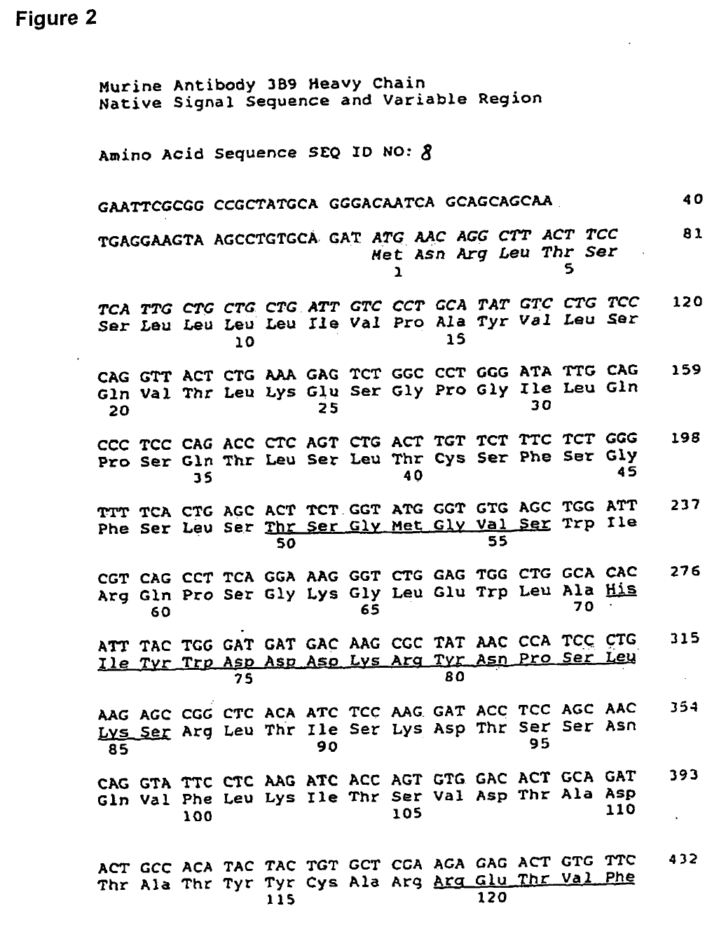

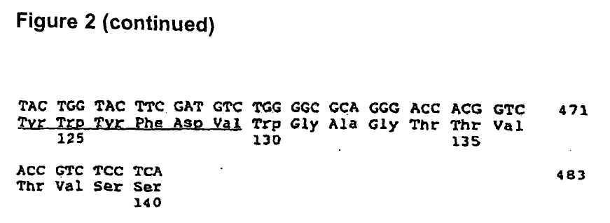

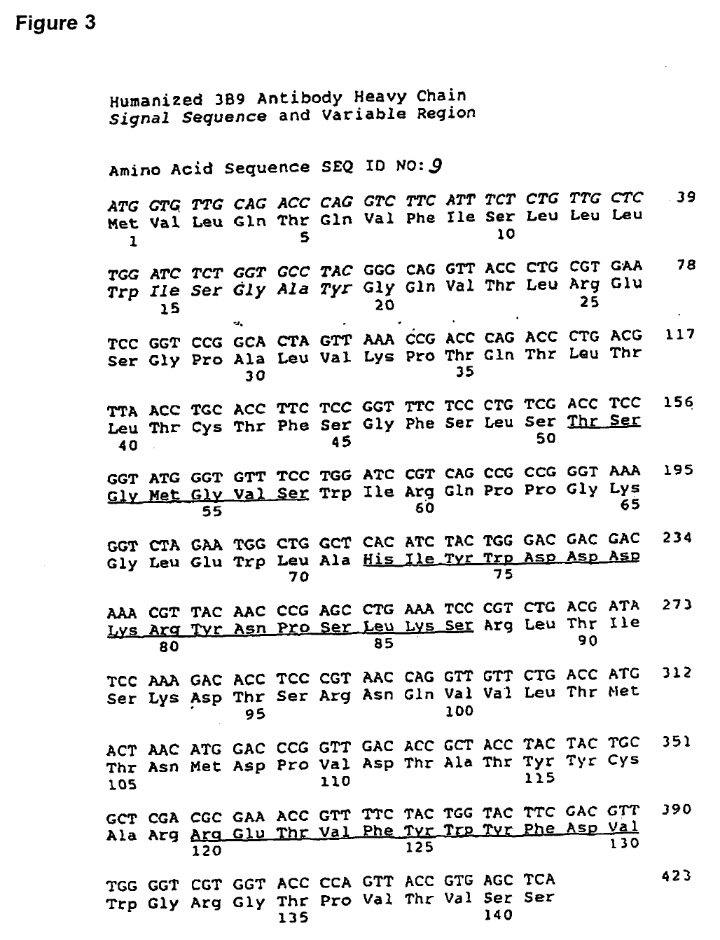

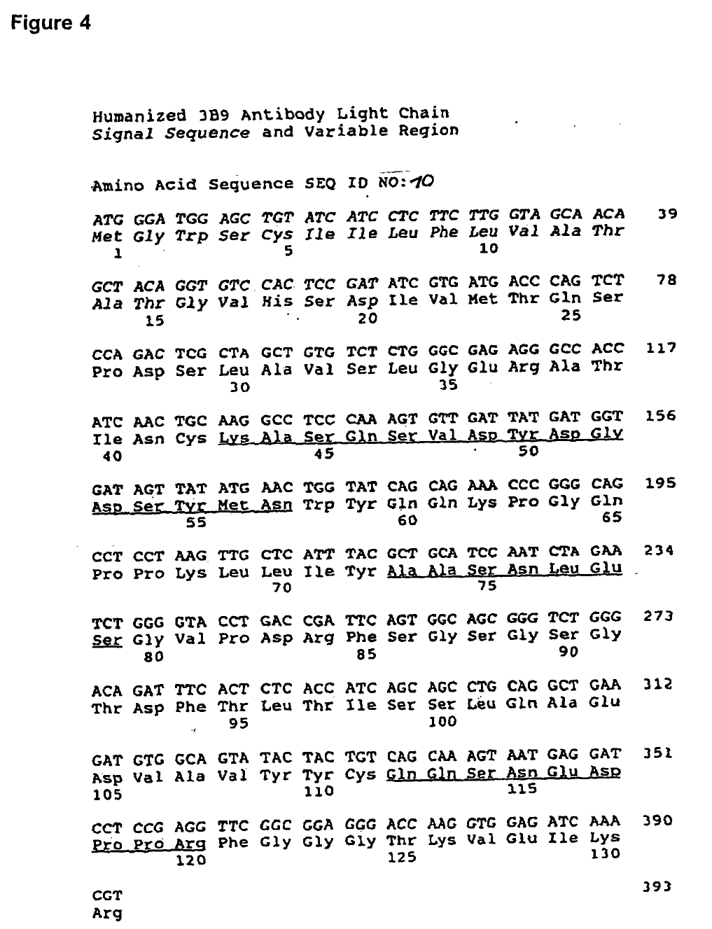

The antibody of the invention is preferably an antibody or an antibody fragment, e.g. a chimeric or humanized antibody derived from the murine antibody 3B9 as described in EP-A-0 730 609. The murine antibody 3B9 comprises the light chain amino acid sequence of SEQ ID NO:7 and the heavy chain amino acid sequence of SEQ ID NO:8 (FIGS. 1 and 2). An especially preferred antibody is the humanized 3B9 antibody as described in EP-A-0 730 609 which comprises the light chain amino acid sequence of SEQ ID NO:9 and the heavy chain amino acid sequence of SEQ ID NO:10 (FIGS. 3 and 4).

A further preferred embodiment relates to the use of an antibody 6A1 produced by the hybridoma cell line ECACC 93100620 as disclosed in EP 0 730 609, or an antibody or an antibody fragment derived therefrom, e.g. a chimeric or humanized antibody. This chimeric or humanized antibody preferably comprises the complementary determining regions of the heavy and/or light chain of the antibody 6A1. Further, the invention refers to an antibody that recognizes the same epitope region of human IL-4 as 6A1, or an antigen-binding fragment thereof, for the manufacture of a medicament for the prevention and/or treatment of cancer.

The antibody may be a complete antibody, e.g. an IgG antibody, or an antigen-binding fragment thereof. Preferably, the antibody is a chimeric or humanized antibody which has human constant domains, e.g. human constant IgG1, IgG2, IgG3 or IgG4 domains. More preferably, the antibody is a humanized antibody which additionally comprises human or substantially human framework regions. Also preferred are antibody fragments, e.g. divalent or monovalent antibody fragments such as F(ab)₂ fragments. On the other hand, the antibody may be a recombinant antibody, e.g. a single chain antibody or a fragment thereof, e.g. an scFv fragment.

In a further embodiment of the present invention, the antibody comprises a further different specific binding component. For example, the antibody or antibody fragment may be a fusion polypeptide with the further component or a bispecific antibody. The antibody may recognize in addition to the human IL-4 also an other antigen, e.g. a further cytokine which is associated with cancer. For example, the antibody or the antigen-binding fragment thereof specifically binds to human IL-4 and human IL-10.

In a still further embodiment, the anti-IL-4 antibody may be used in combination with a further separate antibody which is specific for another cancer-associated antigen, e.g. a further cancer-associated cytokine such as human IL-10.

The anti-IL-4 antibody is preferably administered parenterally, e.g. by injection or infusion. For this purpose, the antibody is formulated as a pharmaceutical composition in a physiologically acceptable carrier, optionally together with physiologically acceptable excipients. The weekly dose is preferably in the range of 0.1 mg/kg to 10 mg/kg, more preferably 1 mg/kg to 5 mg/kg, most preferably about 2 mg/kg. The administration is carried out for a time period sufficient to obtain the desired beneficial effect, e.g. induction of a tumor response to treatment. The antibody therapy should then be maintained for a predetermined period, e.g. several weeks.

The antibody is preferably administered in combination with further anti-tumor therapy, e.g. radiation therapy and/or with at least one further medicament, e.g. a chemotherapeutic agent and/or an anti-tumor antibody. In an especially preferred embodiment, the anti IL-4 antibody is administered in combination with radiation therapy and/or at least one chemotherapeutic agent. In a further especially preferred embodiment, the anti IL-4 antibody is administered together with a further anti-cytokine antibody, e.g. an anti IL-10 antibody in combination with radiation therapy and/or at least one chemotherapeutic agent.

The combination therapy may be administered throughout the whole treatment or an interval thereof. For example, the treatment may comprise a first interval wherein the anti IL-4 antibody, optionally together with a further anti-cytokine antibody, is administered without radiation therapy and/or chemotherapy alone and subsequent intervals wherein (i) the IL-4 antibody, optionally together with a further anti-cytokine antibody, is administered with radiation therapy and/or further medicaments, e.g. chemotherapy and/or (ii) radiation therapy and/or further medicaments are administered without the anti IL-4 antibody.

Alternatively, a first treatment interval may comprise combined therapy and subsequent treatment interval may comprise single therapy, i.e. radiation therapy and/or administration of further medicaments without the anti IL-4 antibody, optionally alternating with combined therapy.

In particular, chemotherapeutic agents which may be used in combination with the monoclonal antibodies of the present invention preferably are antineoplastic compounds. Such compounds included in the present invention comprise, but are not restricted to (i) antimetabolites, such as cytarabine, fludarabine, 5-fluoro-2′-deoxyuridine, gemcitabine, hydroxyurea or methotrexate; (ii) DNA-fragmenting agents, such as bleomycin, (iii) DNA-crosslinking agents, such as chlorambucil, platinum compounds, e.g. cisplatin or oxaliplatin, cyclophosphamide or nitrogen mustard; (iv) intercalating agents such as adriamycin (doxorubicin) or mitoxantrone; (v) protein synthesis inhibitors, such as L-asparaginase, cycloheximide, puromycin or diphteria toxin; (vi) topoisomerase I inhibitors, such as camptothecin or topotecan; (vii) topoisomerase II inhibitors, such as etoposide (VP-16) or teniposide; (viii) microtubule-directed agents, such as colcemide, colchicine, taxanes, e.g. paclitaxel, vinblastine or vincristine; (ix) kinase inhibitors such as flavopiridol, staurosporine or derivatives thereof, e.g. STI571 (CPG 57148B) or UCN-01 (7-hydroxystaurosporine); (x) miscellaneous agents such as thioplatin, PS-341, phenylbutyrate, ET-18-OCH3, or farnesyl transferase inhibitors (L-739749, L-744832); polyphenols such as quercetin, resveratrol, piceatannol, epigallocatechine gallate, theaflavins, flavanols, procyanidins, betulinic acid and derivatives thereof; (xi) hormones such as glucocorticoids or fenretinide; (xii) hormone antagonists, such as tamoxifen, finasteride or LHRH antagonists.

In an especially preferred embodiment of the present invention, the chemotherapeutic agent is selected from the group consisting of platinum compounds, e.g. cisplatin or oxaliplatin, doxorubicin and taxanes, e.g. paclitaxel.

Particularly, the antibodies can be used for the treatment of cancer types which are associated with increased IL-4 expression and/or which are at least partially resistant to apoptosis due to the expression of anti-apoptotic proteins.

Examples of such cancer types comprise neuroblastoma, intestine carcinoma such as rectum carcinoma, colon carcinoma, familiary adenomatous polyposis carcinoma and hereditary non-polyposis colorectal cancer, esophageal carcinoma, labial carcinoma, larynx carcinoma, hypopharynx carcinoma, tongue carcinoma, salivary gland carcinoma, gastric carcinoma, adenocarcinoma, medullary thyroid carcinoma, papillary thyroid carcinoma, follicular thyroid carcinoma, anaplastic thyroid carcinoma, renal carcinoma, kidney parenchym carcinoma, ovarian carcinoma, cervix carcinoma, uterine corpus carcinoma, endometrium carcinoma, chorion carcinoma, pancreatic carcinoma, prostate carcinoma, testis carcinoma, breast carcinoma, bladder carcinoma, melanoma, brain tumors such as glioblastoma, astrocytoma, meningioma, medulloblastoma and peripheral neuroectodermal tumors, Hodgkin lymphoma, non-Hodgkin lymphoma, Burkitt lymphoma, acute lymphatic leukemia (ALL), chronic lymphatic leukemia (CLL), acute myeloid leukemia (AML), chronic myeloid leukemia (CML), adult T-cell leukemia lymphoma, hepatocellular carcinoma, gall bladder carcinoma, bronchial carcinoma, small cell lung carcinoma, non-small cell lung carcinoma, multiple myeloma, basalioma, teratoma, retinoblastoma, choroidal melanoma, seminoma, rhabdomyosarcoma, craniopharyngeoma, osteosarcoma, chondrosarcoma, myosarcoma, liposarcoma, fibrosarcoma, Ewing sarcoma and plasmocytoma.

In a particularly preferred embodiment, the IL-4 antibodies according to the present invention can be used for the treatment of non-lymphoid and non-myeloid, cancers, most preferably epithelial cancers.

Especially preferred examples of cancer types where the use of the IL-4 antibodies according to the present invention is particularly advantageous include all forms of thyroid carcinomas (medullary thyroid carcinoma, papillary thyroid carcinoma, follicular thyroid carcinoma, anaplastic thyroid carcinoma), breast carcinoma, lung carcinoma, prostate carcinoma and colon carcinoma. Most preferably, the IL-4 antibodies are useful for the treatment of thyroid or colon carcinoma, preferably in combination with further therapy as described above. For the treatment of colon carcinoma, IL-4 antibodies are preferably administered together with chemotherapy and/or radiation therapy. For the treatment of thyroid carcinoma, IL-4 antibodies are preferably administered together with IL-10 antibodies and together with chemotherapy and/or radiation therapy.

Furthermore, the IL-4 antibodies according to the present invention are suitable for inducing death of cancer stem cells, e.g. colon cancer stem cells or cancer stem cells in other cancer types as described above. Thus, the antibodies can be used for the treatment of minimal residual disease (MRD) and/or tumor metastasis. The antibodies are preferably administered in combination with further therapy as described above.

The present invention shall be illustrated further by the following example.

### EXAMPLE

The efficacy of the antibodies of the present invention was tested for several cancer types. The anti-IL-4 (α-IL-4) antibody 6A1 (ECACC 92100620) was compared with a commercially available IL-4 antibody from R&D Systems (α-IL-4 R&D).

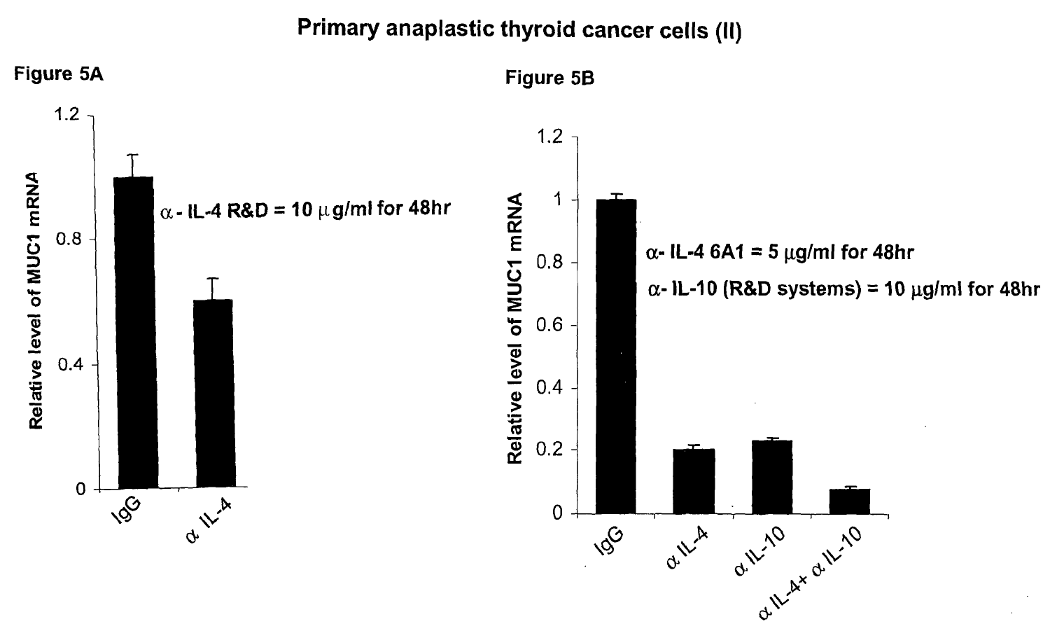

FIG. 5 shows the effect of the respective anti-IL-4 antibodies on the level of MUC1 mRNA in primary anaplastic thyroid cancer cells. MUC1 is an oncoprotein which confers resistance of cancer cells against cytotoxic agents (Yin et al., J. Biol. Chem. 278 (2003), 35458-35466 and Ren et al., Cancer Cell 5 (2004), 163-175). Thus, a decreased level of MUC1 is associated with an increased sensitivity of the cancer cells against chemotherapy and/or radiation.

FIG. 5A shows that treatment of the cancer cells with comparative antibody α-IL-4 R&D (10 μg/ml for 48 hours). The level of MUC1 mRNA is moderately reduced, i.e. to a level of about 60% compared to the control level after administration of non-specific IgG (IgG).

In FIG. 5B, the effect of administration of α-IL-4 6A1 (5 μg/ml for 48 hours) is shown compared to the control (unspecific IgG). The level of MUC1 mRNA is significantly reduced, i.e. to a level of about 20% compared to the control level. Thus, the antibodies of the invention have a higher efficacy compared to other IL-4 antibodies.

Further, FIG. 5B shows the effect of administration of a commercially available IL-10 antibody from R&D Systems (α-IL-10 R&D) and a combination of the anti IL-4 antibody 6A1 and the anti IL-10 antibody. The combined administration of α-IL-4 6A1 and α-IL-10 leads to a further reduction of the level of MUC1 mRNA, i.e. to a level of about less than 10% compared to the control level. Thus, a combination of the antibodies of the invention with IL-10 antibodies has a very high efficacy against thyroid cancer cells.

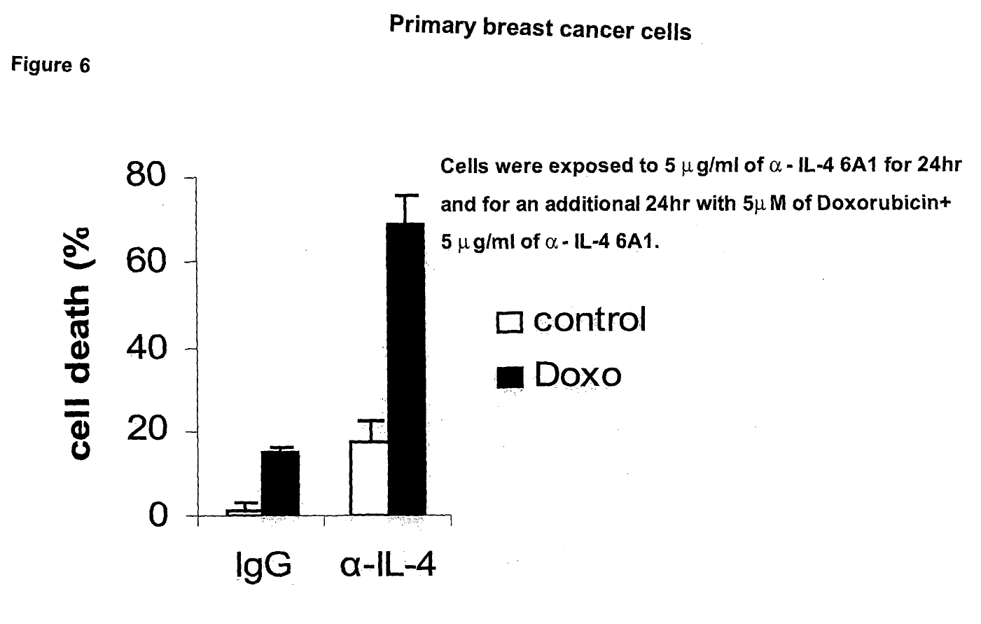

FIG. 6 shows the effect of treating primary breast cancer cells with α-IL-4 6A1 for 24 hours (α-IL-4/control) and for additional 24 hours with 5 μg α-IL-4 6A1 and 5 μmol/l doxorubicin (α-IL-4/doxo). In comparison thereto, the effect of non-specific IgG (IgG) is shown. Combined administration of α-IL-4 6A1 and doxorubicin results in a high percentage of cancer cell death (about 70%).

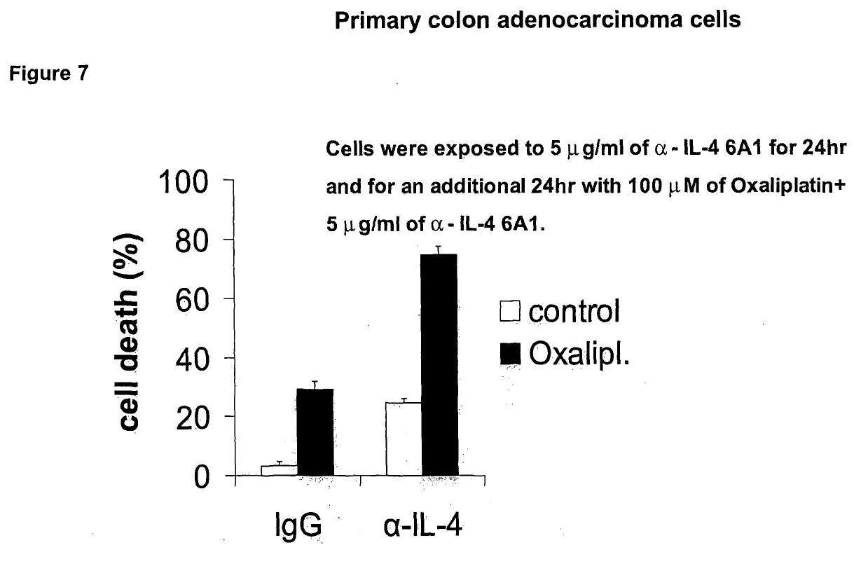

FIG. 7 shows the effect of treatment of primary colon adenocarcinoma cells with the antibody α-IL-4 6A1 (α-IL-4/control) and α-IL-4 6A1 with oxaliplatin (α-IL-4/oxalipl) compared to respective treatment with non-specific IgG (IgG). Cells were exposed to 5 μg/ml of α-IL-4 6A1 for 24 hours and for additional 24 hours with 100 μmol/l oxaliplatin and 5 μg/ml α-IL-4 6A1. Combined administration of α-IL-4 6A1 and oxaliplatin results in a high percentage of cancer cell death (about 70%).

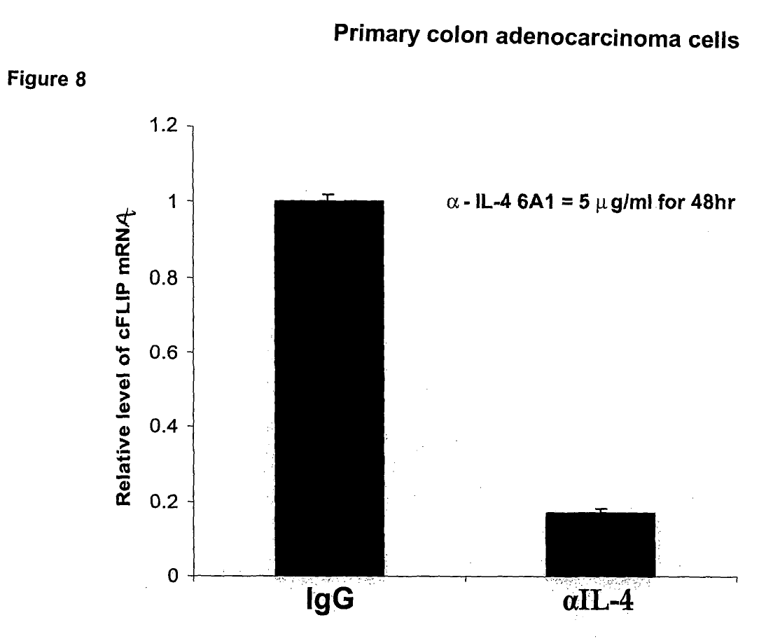

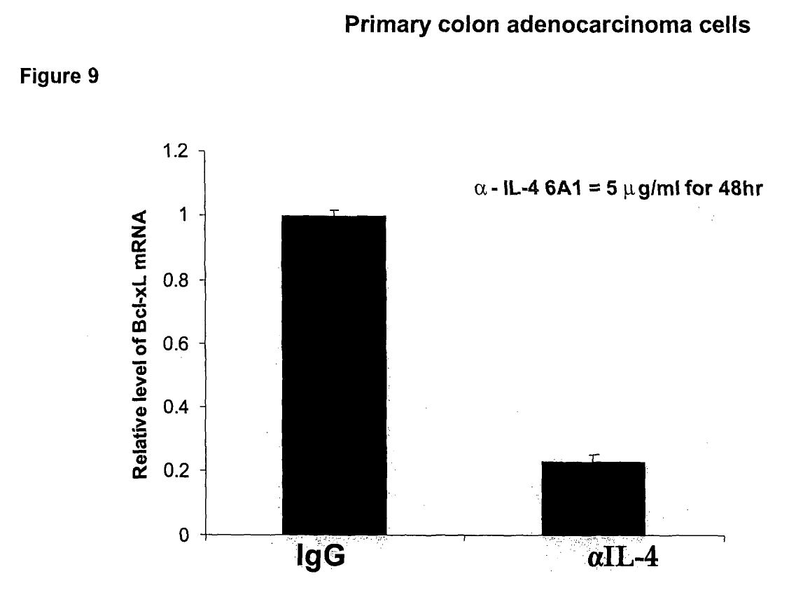

FIGS. 8 and 9 show the effect of treating primary colon adenocarcinoma cells with 5 μg/ml α-IL-4 6A1 for 48 hours on the relative level of cFLIP mRNA (FIG. 8) and Bcl-xL mRNA (FIG. 9). cFLIP and Bcl-xL are anti-apoptotic proteins (Yang et al., Science 275 (1997), 1129-1132; Adams and Cory, Science 281 (1998), 1322-1326; Scaffidi et al., J. Biol. Chem. 274 (1999), 1541-1548; Reed, J. Clin. Oncol. 17 (1999), 2941-2953; Djerbi et al., J. Exp. Med. 190 (1999), 1025-1032; Reed, Nature 387 (1997), 773-776). High levels of these proteins or their mRNAs correlate with a low sensitivity of cancer cells against chemotherapy and/or radiation therapy. It can be gathered from FIGS. 8 and 9 that administration of α-IL-4 6A1 leads to a drastic reduction of mRNA levels and thus to an increased sensitivity of the cancer cells against chemotherapy and/or radiation therapy.

1. Use of an antibody or an antigen-binding fragment specific for human IL-4 for the manufacture of a medicament for the prevention and/or treatment of cancer, wherein the antibody or antibody fragment is characterized by a dissociation constant equal to or less than 2×10⁻¹⁰ M for human IL-4.

2. Use of
(i) an antibody or an antigen-binding fragment thereof specific for human interleukin-4, wherein said monoclonal antibody comprises at least one heavy chain variable region and at least one light chain variable region, wherein the amino acid sequences of the complementarity determining regions (CDRs) of the heavy chain are:
(a) Thr Ser Gly Met Gly Val Ser (SEQ ID No.1);
(b) His Ile Tyr Trp Asp Asp Asp Lys Arg Tyr Asn Pro Ser Leu Lys Ser (SEQ ID No.2);
(c) Arg Glu Thr Val Phe Tyr Trp Tyr Phe Asp Val (SEQ ID No.3); and
(d) a sequence derived by substituting 1, 2 or 3 amino acids of SEQ ID Nos. 1, 2 and/or 3;
and/or the amino acid sequences of the complementarity determining regions (CDRs) of the light chain are:
(a) Lys Ala Ser Gln Ser Val Asp Tyr Asp Gly Asp Ser Tyr Met Asn (SEQ ID No.4);
(b) Ala Ala Ser Asn Leu Glu Ser (SEQ ID No.5);
(c) Gln Gin Ser Asn Glu Asp Pro Pro Thr (Arg) (SEQ ID No.6);
(d) a sequence derived by substituting 1, 2 or 3 amino acids of SEQ ID Nos. 4, 5 and/or 6;
or
(ii) an antibody or an antigen-binding fragment thereof which recognizes the same epitope on human IL-4 as the antibody of (i);
for the manufacture of a medicament for the prevention and/or treatment of cancer.

3. The use according to claim 1 or 2, wherein the antibody is derived from an antibody comprising the light chain amino acid sequence of SEQ ID No 7 and the heavy chain amino acid sequence of SEQ ID No 8.

4. The use according to claim 3, wherein the monoclonal antibody comprises the light chain amino acid sequence of SEQ ID No 9 and the heavy chain amino acid sequence of SEQ ID No 10.

5. Use of
(i) an antibody or an antigen-binding fragment thereof specific for human interleukin-4, wherein the antibody is produced by the hybridoma cell ECACC 93100620 or an antibody or antibody fragment derived therefrom
(ii) an antibody or an antigen-binding fragment thereof which recognizes the same epitope on human IL-4 as the antibody of (i);
for the manufacture of a medicament for the prevention and/or treatment of cancer.

6. The use according to any one of claims 1 to 5, wherein the antibody or an antigen-binding fragment thereof is selected from a chimeric antibody, a partially or fully humanized antibody, a single chain antibody or a fragment thereof.

7. The use according to any one of the preceding claims, wherein the antibody or the antigen-binding fragment comprises in addition to the IL-4 binding component, further different specific binding component.

8. The use according to claim 7, wherein the antibody or the antigen-binding fragment thereof is a fusion polypeptide or a bispecific antibody.

9. The use according to claim 7 or 8, wherein the antibody or the antigen-binding fragment thereof specifically binds to human IL-4 and human IL-10.

10. The use according to any one of the preceding claims in combination with a separate antibody specific to human IL-10.

11. The use according any one of the preceding claims in combination with radiation therapy.

12. The use according to any one of the preceding claims in combination with at least one chemotherapeutic agent.

13. Use according to claim 12, wherein the chemotherapeutic agent is selected from antimetabolites, DNA-fragmenting agents, DNA-crosslinking agents, intercalating agents, protein synthesis inhibitors, topoisomerase I and II inhibitors, microtubule-directed agents, kinase inhibitors, hormones and hormones antagonists.

14. The use according to claim 12 or 13, wherein the chemotherapeutic agent is selected from taxanes, platinum compounds and doxorubicin.

15. Use according to any one of the preceding claims, wherein the medicament additionally comprises pharmaceutical acceptable carriers and/or excipients.

16. Use according to any of the preceding claims, for the prevention and/or treatment of cancer types which are at least partially resistant to apoptosis.

17. Use according to any of the preceding claims, wherein the cancer disease is a epithelial cancer.

18. Use according to claim 17, wherein the cancer disease is selected from the group consisting of thyroid carcinoma, breast carcinoma, lung carcinoma, prostate carcinoma, bladder carcinoma and colon carcinoma.

19. Use according to claim 18, wherein the cancer disease is a thyroid carcinoma, such as a medullary thyroid carcinoma, a papillary thyroid carcinoma, a follicular thyroid carcinoma or a anaplastic thyroid carcinoma.


SEQUENCE LISTING (14 sequences)

SEQ ID NO: 1 - protein, 7 aa, Homo sapiens
  PEPTIDE (1)..(7): CDR of heavy chain
TSGMGVS

SEQ ID NO: 2 - protein, 16 aa, Homo sapiens
  PEPTIDE (1)..(16): CDR of heavy chain
HIYWDDDLRYNPSLKS

SEQ ID NO: 3 - protein, 11 aa, Homo sapiens
  PEPTIDE (1)..(11): CDR of heavy chain
RETVFYWYFDV

SEQ ID NO: 4 - protein, 15 aa, Homo sapiens
  PEPTIDE (1)..(15): CDR of light chain
KASQSVDYDGDSYMN

SEQ ID NO: 5 - protein, 7 aa, Homo sapiens
  PEPTIDE (1)..(7): CDR of light chain
AASNLES

SEQ ID NO: 6 - protein, 10 aa, Homo sapiens
  PEPTIDE (1)..(10): CDR of light chain
QQSNEDPPTR

SEQ ID NO: 7 - DNA, 396 bp, Murinae gen. sp.
  CDS (1)..(396): murine antibody 3B9 light chain
atggagacagacacaatcctgctatgggtgctgctgctctgggttccaggctccactggtgacattgtgctgacccaatctccagcttctttggctgtgtctctagggcagagggccaccatctcctgcaaggccagccaaagtgttgattatgatggtgatagttatatgaactggtaccaacagaaaccaggacagccacccaaactcctcatctatgctgcatccaatctagaatctgggatcccagccaggtttagtggcagtgggtctgggacagacttcaccctcaacatccatcctgtggaggaggaggatgctgcaacctattactgtcagcaaagtaatgaggatcctccgacgttcggtggaggcaccaagctggaaatcaaacgg

SEQ ID NO: 8 - protein, 132 aa, Murinae gen. sp.
METDTILLWVLLLWVPGSTGDIVLTQSPASLAVSLGQRATISCKASQSVDYDGDSYMNWYQQKPGQPPKLLIYAASNLESGIPARFSGSGSGTDFTLNIHPVEEEDAATYYCQQSNEDPPTFGGGTKLEIKR

SEQ ID NO: 9 - DNA, 483 bp, Murinae gen. sp.
  CDS (64)..(483): murine antibody 3B9 heavy chain
gaattcgcggccgctatgcagggacaatcagcagcagcaatgaggaagtaagcctgtgcagatatgaacaggcttacttcctcattgctgctgctgattgtccctgcatatgtcctgtcccaggttactctgaaagagtctggccctgggatattgcagccctcccagaccctcagtctgacttgttctttctctgggttttcactgagcacttctggtatgggtgtgagctggattcgtcagccttcaggaaagggtctggagtggctggcacacatttactgggatgatgacaagcgctataacccatccctgaagagccggctcacaatctccaaggatacctccagcaaccaggtattcctcaagatcaccagtgtggacactgcagatactgccacatactactgtgctcgaagagagactgtgttctactggtacttcgatgtctggggcgcagggaccacggtcaccgtctcctca

SEQ ID NO: 10 - protein, 140 aa, Murinae gen. sp.
MNRLTSSLLLLIVPAYVLSQVTLKESGPGILQPSQTLSLTCSFSGFSLSTSGMGVSWIRQPSGKGLEWLAHIYWDDDKRYNPSLKSRLTISKDTSSNQVFLKITSVDTADTATYYCARRETVFYWYFDVWGAGTTVTVSS

SEQ ID NO: 11 - DNA, 423 bp, Artificial
  humanized 3B9 antibody heavy chain
atggtgttgcagacccaggtcttcatttctctgttgctctggatctctggtgcctacgggcaggttaccctgcgtgaatccggtccggcactagttaaaccgacccagaccctgacgttaacctgcaccttctccggtttctccctgtcgacctccggtatgggtgtttcctggatccgtcagccgccgggtaaaggtctagaatggctggctcacatctactgggacgacgacaaacgttacaacccgagcctgaaatcccgtctgacgatatccaaagacacctcccgtaaccaggttgttctgaccatgactaacatggacccggttgacaccgctacctactactgcgctcgacgcgaaaccgttttctactggtacttcgacgtttggggtcgtggtaccccagttaccgtgagctca

SEQ ID NO: 12 - protein, 141 aa, Artificial
  Synthetic Construct
MVLQTQVFISLLLWISGAYGQVTLRESGPALVKPTQTLTLTCTFSGFSLSTSGMGVSWIRQPPGKGLEWLAHIYWDDDKRYNPSLKSRLTISKDTSRNQVVLTMTNMDPVDTATYYCARRETVFYWYFDVWGRGTPVTVSS

SEQ ID NO: 13 - DNA, 393 bp, Artificial
  humanized 3B9 antibody light chain
atgggatggagctgtatcatcctcttcttggtagcaacagctacaggtgtccactccgatatcgtgatgacccagtctccagactcgctagctgtgtctctgggcgagagggccaccatcaactgcaaggcctcccaaagtgttgattatgatggtgatagttatatgaactggtatcagcagaaacccgggcagcctcctaagttgctcatttacgctgcatccaatctagaatctggggtacctgaccgattcagtggcagcgggtctgggacagatttcactctcaccatcagcagcctgcaggctgaagatgtggcagtatactactgtcagcaaagtaatgaggatcctccgaggttcggcggagggaccaaggtggagatcaaacgt

SEQ ID NO: 14 - protein, 131 aa, Artificial
  Synthetic Construct
MGWSCIILFLVATATGVHSDIVMTQSPDSLAVSLGERATINCKASQSVDYDGDSYMNWYQQKPGQPPKLLIYAASNLESGVPDRFSGSGSGTDFTLTISSLQAEDVAVYYCQQSNEDPPRFGGGTKVEIKR